(19) United States
(12) Patent Application Publication
(10) Pub. No.: US 2026/0077152 A1
(43) Pub. Date: Mar. 19, 2026

(54) SLEEP CARE DEVICE

(71) Applicant: SLEEPWAVE INC., Seoul (KR)

(72) Inventors: Jasoo CHO, Seoul (KR); Yangtae KWON, Seoul (KR); Jeongkeun CHOI, Seoul (KR)

(73) Assignee: SLEEPWAVE INC., Seoul (KR)

(21) Appl. No.: 19/108,608

(22) PCT Filed: Dec. 26, 2022
(86) PCT No.: PCT/KR2022/021273

§ 371 Date: Mar. 4, 2025

(30) Foreign Application Priority Data
Sep. 8, 2022 (KR) 10-2022-0114404

(51) Int. Cl. A61M 21/02; A61M 21/00

(52) U.S. Cl. CPC A61M 21/02; A61M 2021/0027; A61M 2205/3584; A61M 2205/8206; A61M 2205/8256; A61M 2230/10

(57) ABSTRACT

A sleep care device is disclosed. The sleep care device is mounted on the ear of a user, is not easily separated from the user's body while the user is in a sleep state, and can reliably measure brain wave signals. The sleep care device according to an example embodiment of the present invention includes a housing, an ear hook, and an ear tip. The ear hook is connected to the housing to form a closed loop, is placed between the user's head and the auricle, and includes an electrode having electrical conductivity. The ear tip is connected to the housing, is placed at the entrance to the external auditory canal, and has electrical conductivity. In addition, the ear hook and the ear tip are made of a material having flexibility and elasticity, and measure the user's brain waves.

### TECHNICAL FIELD

The disclosure relates to a sleep care device that measures user's brain wave signals to help the user fall asleep and remain in a deep sleep.

### BACKGROUND ART

It is very important to maintain a good-quality sleep status in terms of managing a person's health. Therefore, people put a lot of effort into getting a good night's sleep. In this regard, a method of collecting human bio-signals to monitor sleep status and induce deep sleep through appropriate stimulation may be considered.

Biological signals are signals in the form of electrical potential or current generated from human muscle or nerve cells, and may be obtained by collecting and analyzing changes in electrical signals detected through electrodes attached to the human body.

The related art biological signal measuring device has detected bio-signals by attaching wet electrodes to a user's body.

A wet electrode is an electrode that is made of a conductive metal and electrically interfaces with a user's skin through a hydrogel.

Wet electrodes using hydrogels are advantageous for measuring bio-signals because they reduce the influence of contact impedance on microscopic bio-signals by relatively lowering the contact impedance between the electrodes and the user's skin. Using the wet electrodes in this way allows for an acquisition of relatively uniform and stable bio-signals.

However, wet electrodes are difficult to use repeatedly and are not easy to store, making it substantially impossible to use the wet electrodes at home. Furthermore, it is undesirable in consideration of user convenience to use the wet electrodes for collecting sleep signals due to the user's discomfort with wearing the wet electrodes.

On the other hand, dry electrodes are easy to store but are uncomfortable to wear due to the coldness and hardness of metal, and the contact impedance, which is electrical resistance between the electrodes and the skin, is high, making it difficult to collect high-quality signals.

To solve these problems, Korean Patent Publication No. 10-2020-0056744 has proposed a collection of bio-signals by forming electrodes made of silicon material on a wearable device. However, the Korean Patent Publication application has a problem in that the wearable device is easily detached from a user's body. In particular, the user may not easily notice when the wearable device is detached during sleeping, which may cause a problem with reliability of measured bio-signals.

### DISCLOSURE OF INVENTION

### Technical Problem

An aspect of the disclosure is to provide a sleep care device that is not easily detached from a user's body during the user's sleep and is capable of measuring reliable brain wave (electroencephalogram (EEG)) signals.

### Solution to Problem

The above-described aspect of the disclosure is achieved by the specific contents described below.

A sleep care device according to an embodiment of the disclosure is a sleep care device that is worn on a user's ear, and includes a housing, an ear hook, and an eartip. The housing forms an exterior of the sleep care device. The ear hook is connected to the housing to form a closed loop, is arranged between the user's head and an auricle, and includes an electrode having electrical conductivity. The eartip is connected to the housing, is arranged at an entrance of an external auditory canal, and has electrical conductivity. The ear hook and the eartip are made of a material having flexibility and elasticity and measure the user's electroencephalogram (EEG).

Specifically, the ear hook includes a first electrode and a second electrode having electrical conductivity, and a connection portion arranged between the first electrode and the second electrode.

Specifically, the first electrode or the second electrode is formed in an arcuate shape.

Specifically, the connection portion is formed in a wavy shape.

Specifically, a plurality of fastening holes are formed in each of one end portion of the first electrode and another end portion of the second electrode.

Specifically, the plurality of fastening holes include at least one fastening protrusion hole and a fastening screw hole.

Specifically, the housing includes an upper housing and a lower housing. A fastening protrusion formed on one side of the lower housing is arranged by being inserted into the fastening protrusion hole formed in the one end portion of the first electrode. A fastening protrusion formed on another side of the lower housing is arranged by being inserted into the fastening protrusion hole formed in the another end portion of the second electrode.

Specifically, the fastening screw hole formed in the one end portion of the first electrode is placed in a fastening screw groove formed in one side of the lower housing. The fastening screw hole formed in the another end portion of the second electrode is placed in a fastening screw groove formed in another side of the lower housing.

Specifically, a substrate is arranged inside the housing. One end portion of the first electrode and another end portion of the second electrode are electrically connected to the substrate by a metal clip mounted on the substrate and having electrical conductivity.

Specifically, the eartip includes a tip body connected to the housing and a tip ring connected to the tip body.

Specifically, the tip body includes a body groove and a body hole. The body groove is a groove formed with a preset depth in one surface of the tip body, and the body hole is a hole penetrating from a bottom surface of the body groove to another surface of the tip body.

Specifically, one end portion of an electrical terminal electrically connected to the substrate is forcibly fitted into the body groove.

Specifically, a substrate is arranged between the upper housing and the lower housing, and an inner housing is arranged between the lower housing and the substrate. An upper end portion of a speaker receiving part formed in the inner housing is forcibly fitted into the body groove. The upper end portion of the speaker receiving part is arranged below the body hole.

Specifically, a sound pressure hole communicating with the body hole is formed in the upper end portion of the speaker receiving part.

Specifically, the tip ring has a ring shape and is arranged as two tip rings. The two tip rings are respectively connected to one side and another side of the tip body.

Specifically, an electrode of the ear hook and the eartip are formed by mixing plate-shaped silver (Ag) powder, spherical silver (Ag) powder, or silver flakes with a silicone polymer.

Specifically, the upper housing is arranged toward an opposite side of the user's head, and the lower housing is arranged to oppose the head with the eartip located therebetween. The lower housing includes a speaker receiving part hole through which an upper end portion of a speaker receiving part formed in the inner housing passes, and a tip terminal hole through which an electrical terminal electrically connected to the substrate passes.

### Advantageous Effects of Invention

A sleep care device according to an embodiment of the disclosure has an effect of being suppressed from being easily separated from a user's body during the user's sleep state and measuring reliable EEG signals.

The sleep care device according to an embodiment of the disclosure has an effect of enabling the user to feel comfortable wearing the sleep care device while sleeping.

The sleep care device according to an embodiment of the disclosure has an effect of being used by plural family members and other individuals without needing to replace the ear hook and the eartip depending on users.

### BRIEF DESCRIPTION OF DRAWINGS

FIG. 1 is a front perspective view of a sleep care device according to an embodiment of the disclosure.

FIG. 2 is a rear perspective view of the sleep care device of FIG. 1.

FIG. 3 is a view of a state in which the sleep care device of FIG. 1 is worn on a user's ear.

FIG. 4 is an exploded perspective view of the sleep care device of FIG. 1.

FIG. 5 is a plan view of an ear hook.

FIG. 6 is a bottom view of the ear hook.

FIG. 7 is a view of a part A of FIG. 6.

FIG. 8 is a bottom perspective view of an eartip.

FIG. 9 is a plan view of the eartip.

FIG. 10 is a view of a state in which the ear hook and the eartip are removed from the sleep care device of FIG. 1.

FIG. 11 is a view of an upper housing. (a) of FIG. 11 is a view of an outer surface of the upper housing 32, and (b) of FIG. 11 is a view of an inner surface of the upper housing 32.

FIG. 12 is a plan view of a lower housing.

FIG. 13 is a bottom view of the lower housing.

FIG. 14 is a view of a state before the ear hook is coupled to the lower housing.

FIG. 15 is a view of a state in which the ear hook is coupled to the lower housing.

FIG. 16 is a view of a state in which the lower housing is removed from FIG. 10.

FIG. 17 is a perspective view of an inner housing.

FIG. 18 is a perspective view of a substrate.

FIG. 19 is a perspective view of a state in which the substrate is arranged in the upper housing.

FIG. 20 is a cross-sectional view of the sleep care device of FIG. 1 cut from the lower housing side toward the upper housing side.

FIG. 21 is a cross-sectional view of a state in which the substrate and one end portion of the first electrode are electrically connected by a metal clip.

FIG. 22 is a view of components (hardware) of the disclosure mounted on or electrically connected to the substrate.

FIG. 23 is a block diagram of the sleep care device of FIG. 1.

FIG. 24 is a view of EEG measured by the sleep care device of FIG. 1.

FIG. 25 compares EEG measured from an ear and EEG measured from scalp using the sleep care device shown in FIG. 1.

FIG. 26 is a view of an embodiment in which the sleep care device of FIG. 1 is placed in a charging case.

### MODE FOR THE INVENTION

Hereinafter, embodiments of the disclosure will be described in detail with reference to the accompanying drawings. A detailed description of components of the disclosure that may be clearly understood and easily reproduced by a person skilled in the art using the related art will be omitted, not to obscure the gist of the disclosure.

The related art bio-signal measuring devices using wet electrodes have the problem with substantially impractical use at home due to the difficulty of repeated use of the wet electrodes and inconvenient storage of the wet electrodes, and also have a problem with undesirable use of the wet electrodes for collecting sleep signals, in consideration of user convenience, due to discomfort with wearing the wet electrodes.

The related art bio-signal measuring devices using dry electrodes have problems that the dry electrodes are easy to store but are uncomfortable to wear due to the coldness and hardness of metal, the contact impedance between the dry electrodes and the skin is high, and the dry electrodes are easily detached from the user who is sleeping, making it difficult to collect high-quality signals.

A sleep care device described in this specification is characterized in that a user can feel comfortable while wearing the sleep care device in a sleep status, the sleep care device is not easily detached from the user's body, and there is no need to replace an ear hook and an eartip depending on users.

Hereinafter, a sleep care device according to an embodiment of the disclosure will be described.

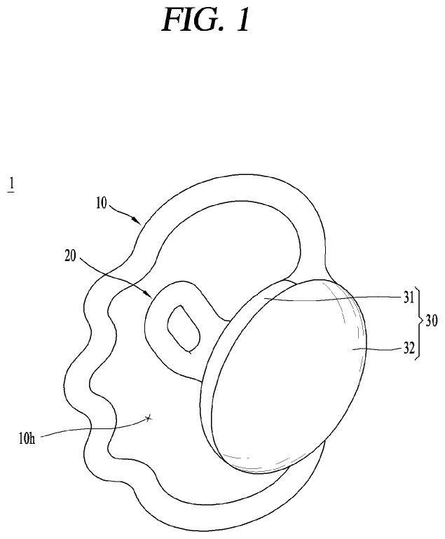

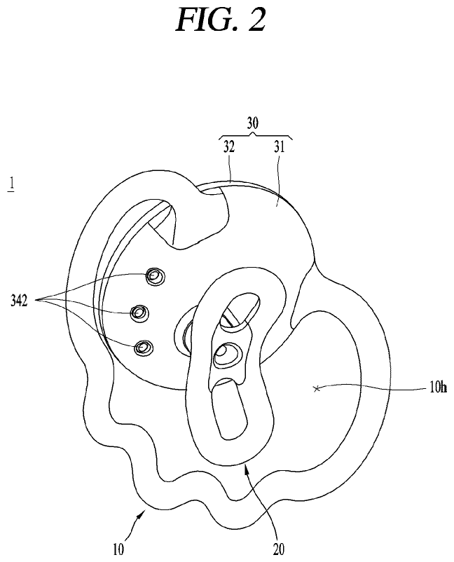

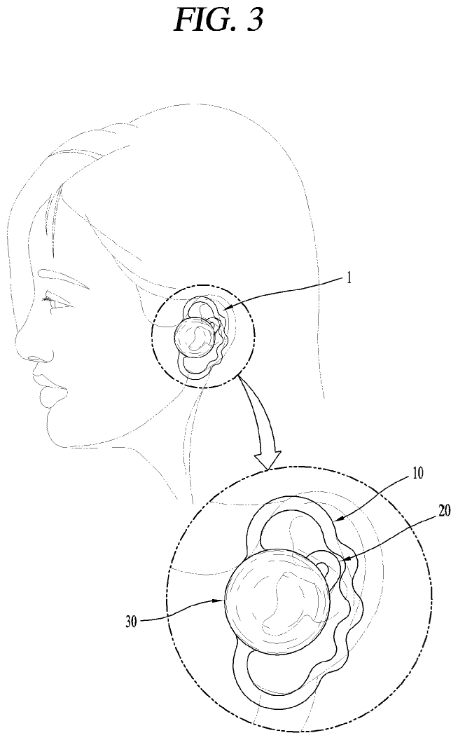

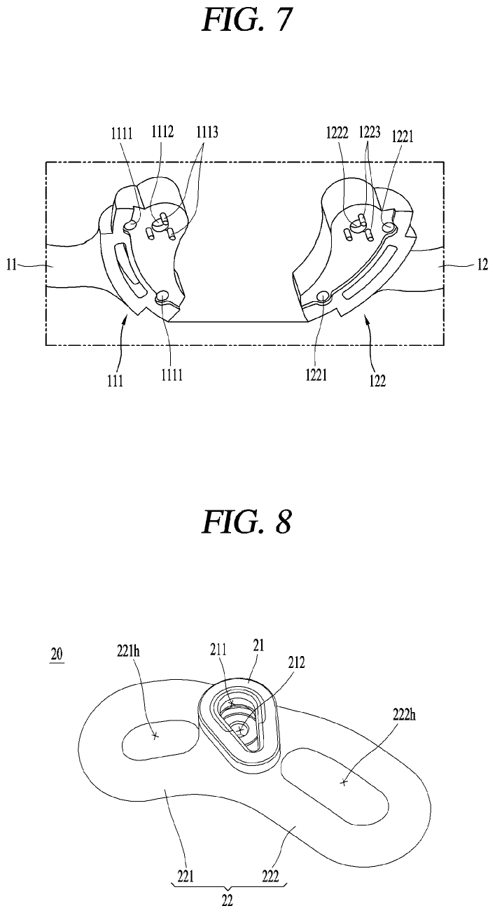

Referring to FIGS. 1 to 7, a sleep care device 1 according to an embodiment of the disclosure includes an ear hook 10, an eartip 20, and a housing 30.

The ear hook 10 serves to secure the sleep care device 1 according to the disclosure to an ear. The ear hook 10 suppresses the sleep care device 1 according to the disclosure from being easily detached from the user's ear.

The ear hook 10 is formed in the shape of a string (or line) having a preset length.

In some embodiments, the ear hook 10 includes one end portion 111 and another end portion 122 opposite to the one end portion 111, and is formed in a loop (open loop) shape having a preset length extending from the one end portion 111 to the another end portion 122.

The ear hook 10 also has a preset cross-sectional diameter. Here, the cross-section indicates a cut surface when the ear hook 10 is cut in a direction orthogonal to a longitudinal direction of the ear hook 10. The shape of the cross-section may be circular, elliptical, or polygonal.

The ear hook 10 is connected to the housing 30.

The ear hook 10 is connected to the housing 30 to form a closed loop. An auricle is inserted and placed into an opening 10h inside the closed loop.

In some embodiments, the one end portion 111 of the ear hook 10 is connected to one side 311 of the housing 30, and the another end portion 122 of the ear hook 10 is connected to another side 312 of the housing 30. Here, the another side 312 of the housing 30 corresponds to a portion of the housing 30 which is adjacent to the one side 311 of the housing 30 and spaced apart from the one side 311 by a preset distance. The one side 311 of the housing 30 indicates one side 311 of a lower housing 31 to be described later, and the another side 312 of the housing 30 indicates another side 312 of the lower housing 31.

The eartip 20 to be described later is arranged at an entrance of an external auditory canal (concha) of the ear when the sleep care device 1 is mounted on the user's ear. The housing 30 connected to the eartip 20 is arranged to oppose the ear with the eartip 20 located therebetween. The ear hook 10 connected to the housing 30 forms a closed loop together with the housing 30. An auricle is inserted into the opening 10h inside the closed loop, and the ear hook 10 is arranged between the head and the auricle to surround the auricle. For this reason, the ear hook 10 serves to fix the sleep care device 1 of the disclosure so that the sleep care device 1 is not easily detached from the user's ear.

The ear hook 10 is formed of a material (or substance) having flexibility and elasticity. For example, the ear hook 10 may be formed of a silicone material having flexibility and elasticity. Here, flexibility refers to the property of being elastic, bendable, and flexible. Elasticity refers to the property of an object (the ear hook 10) deformed by an external force to return to its original state when the external force is removed.

The ear hook 10 is formed of a flexible and elastic material, and is arranged between the head and the auricle to maintain contact (close contact) state with the head and/or the auricle.

The ear hook 10 includes a first electrode 11, a second electrode 12, and a connection portion 13.

The first electrode 11 includes one end portion 111 and another end portion 112 opposite to the one end portion 111. The first electrode 11 has a preset length extending from the one end portion 111 to the another end portion 112.

The one end portion 111 of the first electrode 11 corresponds to the one end portion 111 of the ear hook 10 and is connected to the one side 311 of the housing 30. The another end portion 112 of the first electrode 11 is connected to the connection portion 13 (for example, one end portion 131 of the connection portion 13).

The first electrode 11 is formed to have the preset length. Therefore, even though a portion of the first electrode 11 does not come into contact with the head and/or the auricle, another portion of the first electrode 11 may come into contact with the head and/or the auricle. Accordingly, the first electrode 11 is kept in contact with the head and/or the auricle when the sleep care device 1 of the disclosure is mounted on the ear.

The first electrode 11 is formed in an arcuate shape to surround the auricle. Here, the auricle may refer to, for example, an auricle, which corresponds to an upper part of the ear.

The one end portion 111 of the ear hook 10, that is, the one end portion 111 of the first electrode 11, is connected to the one side 311 of the housing 30. Specifically, the one end portion 111 of the first electrode 11 is coupled to the one side 311 of the lower housing 31 to be described later.

The one end portion 111 of the first electrode 11 may be formed to correspond to a shape of a portion of the lower housing 31 to which the one end portion 111 is coupled. However, the one end portion 111 of the first electrode 11 is not limited to a certain shape and may be formed in various shapes.

The one end portion 111 of the first electrode 11 includes a plurality of fastening holes.

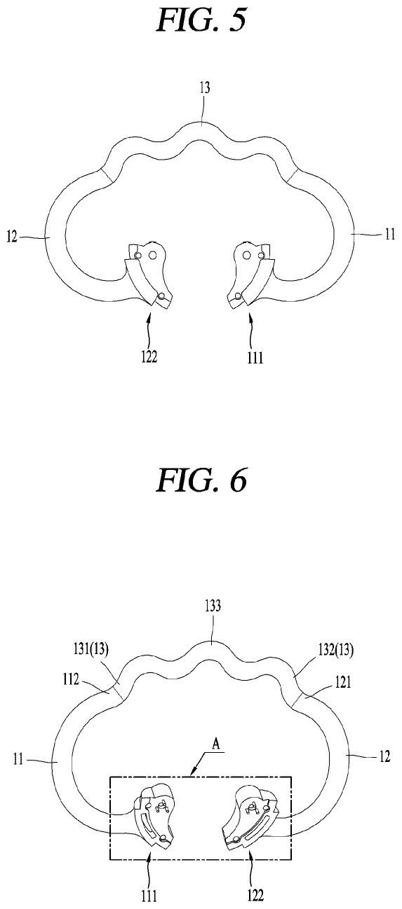

The plurality of fastening holes include at least one fastening protrusion hole 1111 and a fastening screw hole 1112. In FIGS. 5 to 7, two fastening protrusion holes 1111 are formed, and one fastening screw hole 1112 is formed.

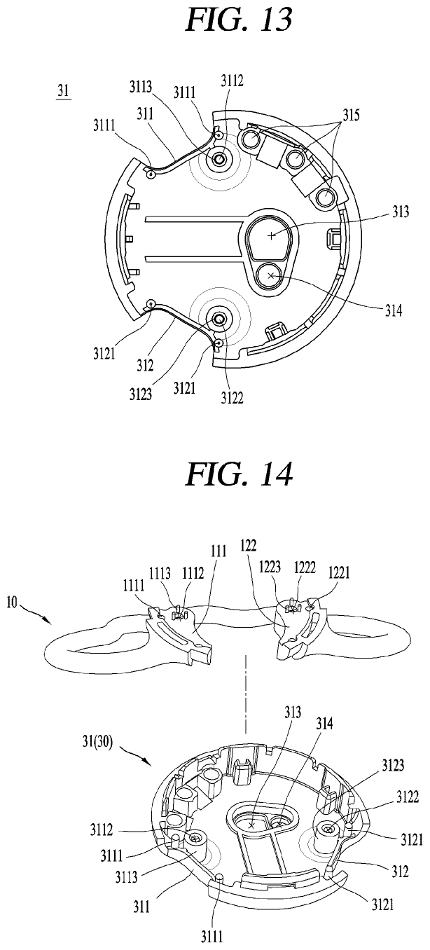

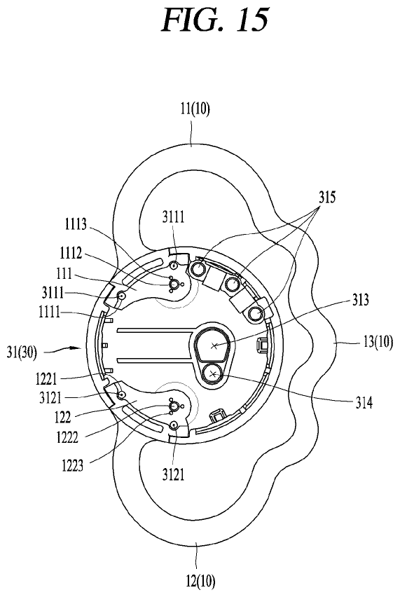

Fastening protrusions 3111 (see FIGS. 13 to 15) that are formed on the one side 311 of the lower housing 31 are inserted into the fastening protrusion holes 1111. Accordingly, the one end portion 111 of the ear hook 10 (specifically, the one end portion 111 of the first electrode 11) is fixed to the lower housing 31. Although not shown, according to another embodiment of the disclosure, at least one fastening protrusion may be formed on the one end portion 111 of the first electrode 11 and a fastening protrusion hole may be formed in the one side 311 of the lower housing 31, so that the one end portion 111 of the first electrode 11 and the one side 311 of the lower housing 31 are coupled to each other.

The fastening screw hole 1112 is seated in a fastening screw groove 3112 (see FIGS. 13 to 15) formed in the one side 311 of the lower housing 31.

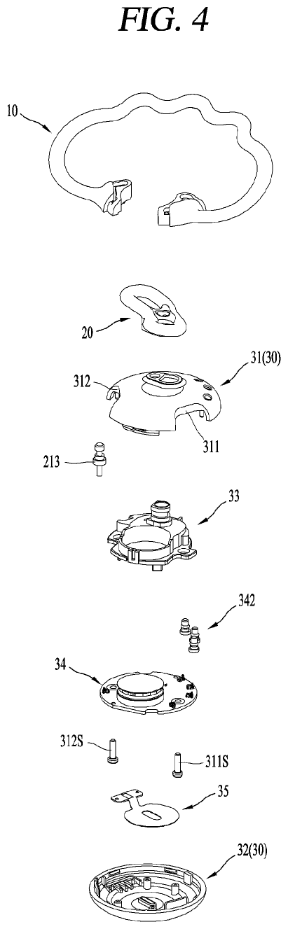

A fastening screw 311S (see FIG. 4) passes through a fastening screw hole 34h1 (see FIG. 19) formed in a substrate 34 to be described later and the fastening screw hole 1112 formed in the one end portion 111 of the first electrode 11, and is fixedly inserted into the fastening screw groove 3112 formed in the one side 311 of the lower housing 31. Accordingly, the substrate 34 and the one end portion 111 of the first electrode 11 are fixed to the one side 311 of the lower housing 31.

A plurality of support protrusions 1113 may be formed on a circumference of the fastening screw hole 1112 at the one end portion 111 of the first electrode 11.

The plurality of support protrusions 1113 protrude from the one end portion 111 of the first electrode 11 toward the substrate 34 and are arranged spaced apart by preset intervals.

The plurality of support protrusions 1113 support the substrate 34 and make the one end portion 111 of the first electrode 11 spaced apart from the substrate 34. Accordingly, the one end portion 111 of the first electrode 11 comes into contact with a certain electric circuit formed on the substrate 34 through a metal clip 341 to be described later, without direct contact with the substrate 34.

The second electrode 12 includes one end portion 121 and another end portion 122 opposite to the one end portion 121. The second electrode 12 has a preset length extending from the one end portion 121 to the another end portion 122.

The one end portion 121 of the second electrode 12 is connected to the connection portion 13 (specifically, another end portion 132 of the connection portion 13). The another end portion 122 of the second electrode 12 corresponds to the another end portion 122 of the ear hook 10 and is connected to the another side 312 of the housing 30.

The second electrode 12 is formed to have the preset length. Therefore, even though a portion of the second electrode 12 does not come into contact with the head and/or the auricle (or ear lobe), another portion of the second electrode 12 comes into contact with the head and/or the auricle (or ear lobe). Accordingly, the second electrode 12 is kept in contact with the head and/or the auricle (or ear lobe) when the sleep care device 1 of the disclosure is mounted on the ear.

The second electrode 12 is formed in an arcuate shape to surround the auricle (or ear lobe). Here, the auricle (or ear lobe) may refer to, for example, an auricle (or ear lobe) corresponding to a lower portion of the ear.

The another end portion 122 of the ear hook 10, for example, the another end portion 122 of the second electrode 12, is connected to the another side 312 of the housing 30. In some embodiments, the another end portion 122 of the second electrode 12 is coupled to the another side 312 of the lower housing 31 to be described later.

The another end portion 122 of the second electrode 12 may be formed to correspond to a shape of a portion of the lower housing 31 to which the another end portion 122 is coupled. However, the another end portion 122 of the second electrode 12 is not limited to a certain shape and may be formed in various shapes.

The another end portion 122 of the second electrode 12 includes a plurality of fastening holes.

The plurality of fastening holes formed in the another end portion 122 of the second electrode 12 include at least one fastening protrusion hole 1221 and a fastening screw hole 1222. In FIGS. 5 to 7, two fastening protrusion holes 1221 are formed, and one fastening screw hole 1222 is formed.

Fastening protrusions 3121 (see FIGS. 13 to 15) that are formed on the another side 312 of the lower housing 31 are inserted into the fastening protrusion holes 1221 formed in the another end portion 122 of the second electrode 12. Accordingly, the another end portion 122 of the ear hook 10 (specifically, the another end portion 122 of the second electrode 12) is fixed to the lower housing 31. Although not shown, according to another embodiment of the disclosure, at least one fastening protrusion may be formed on the another end portion 122 of the second electrode 12 and a fastening protrusion hole may be formed in the another side 312 of the lower housing 31, so that the another end portion 122 of the second electrode 12 and the another side 312 of the lower housing 31 are coupled to each other.

The fastening screw hole 1222 formed in the another end portion 122 of the second electrode 12 is seated in a fastening screw groove 3122 (see FIGS. 13 to 15) formed in the another side 312 of the lower housing 31.

A fastening screw 312S (see FIG. 4) passes through a fastening screw hole 34h2 (see FIG. 19) formed in the substrate 34 and the fastening screw hole 1222 formed in the another end portion 122 of the second electrode 12, and is fixedly inserted into the fastening screw groove 3122 formed in the another side 312 of the lower housing 31. Accordingly, the substrate 34 and the another end portion 122 of the second electrode 12 are fixed to the another side 312 of the lower housing 31.

A plurality of support protrusions 1223 may be formed on a circumference of the fastening screw hole 1222 at the another end portion 122 of the second electrode 12.

The plurality of support protrusions 1223 formed on the another end portion 122 of the second electrode 12 protrude from the another end portion 122 of the second electrode 12 toward the substrate 34 and are arranged spaced apart by preset intervals.

The plurality of support protrusions 1223 formed on the another end portion 122 of the second electrode 12 support the substrate 34 and make the another end portion 122 of the second electrode 12 spaced apart from the substrate 34. Accordingly, the another end portion 122 of the second electrode 12 comes into contact with a certain electric circuit formed on the substrate 34 through a metal clip 341 to be described later, without direct contact with the substrate 34.

The first electrode 11 and the second electrode 12 have electrical conductivity.

The first electrode 11 and the second electrode 12 are formed of a material having flexibility and elasticity. For example, the first electrode 11 and the second electrode 12 may be formed of a silicone material having flexibility and elasticity, and may be formed by including a metal powder having electrical conductivity in the silicone material.

Specifically, the first electrode 11 and the second electrode 12 may be formed by mixing plate-shaped silver (Ag) powder, spherical silver (Ag) powder, and/or silver flakes into a silicone polymer. Accordingly, the first electrode 11 and the second electrode 12 have electrical conductivity while maintaining the characteristics of the silicon material. The first electrode 11 and the second electrode 12 each have a resistance value of 10 Ω (ohm) or less.

The first electrode 11 and the second electrode 12 may sense and measure brain wave signals. Here, electroencephalogram (EEG) is an electrical signal generated by neural activity in the brain. EEG is also called electrocorticogram.

The first electrode 11 and the second electrode 12 serve as electrodes. For example, the first electrode 11 may be a reference electrode, and the second electrode 12 may be an earth electrode (bias). The eartip 20 to be described later may be a measuring electrode.

The first electrode 11 and the second electrode 12 are formed of a flexible and elastic material, and are arranged between the head and the auricle to maintain contact (close contact) state with the head and/or the auricle. The first electrode 11 and the second electrode 12 may include a metal powder having electrical conductivity, thus functioning as electrodes.

The one end portion 111 of the first electrode 11 is in contact with a metal clip 341 (this may be referred to as a ‘first metal clip 341a’), which is mounted on the substrate 34 and has electrical conductivity (see FIGS. 19 and 21).

The first metal clip 341a is mounted (fixed) to the electric circuit of the substrate 34 (see FIG. 21).

The first metal clip 341a is arranged between the one end portion 111 of the first electrode 11 and the substrate 34, electrically connecting the one end portion 111 of the first electrode 11 and the electric circuit of the substrate 34.

Specifically, one side (e.g., an upper side) of the first metal clip 341a is in contact with the one end portion 111 of the first electrode 11, and another side (e.g., a lower side) of the first metal clip 341a is in contact with the electric circuit of the substrate 34.

Accordingly, an electric signal (specifically, EEG) measured by the first electrode 11 is transmitted to the substrate 34 via the one end portion 111 of the first electrode 11 and the first metal clip 341a, and is stored in a memory arranged in the substrate 34 or processed into data for output or communication by a signal processing unit.

The another end portion 122 of the second electrode 12 is in contact with the metal clip 341 (this may be referred to as a ‘second metal clip 341b’), which is mounted on the substrate 34 and has electrical conductivity (see FIG. 19).

The second metal clip 341b is mounted (fixed) to the electric circuit of the substrate 34.

The second metal clip 341b is arranged between the another end portion 122 of the second electrode 12 and the substrate 34, electrically connecting the another end portion 122 of the second electrode 12 and the electric circuit of the substrate 34.

Specifically, one side (e.g., an upper portion) of the second metal clip 341b is in contact with the another end portion 122 of the second electrode 12, and another side (e.g., a lower portion) of the second metal clip 341 is in contact with the electric circuit of the substrate 34.

Accordingly, an electric signal (specifically, EEG) measured by the second electrode 12 is transmitted to the substrate 34 via the another end portion 122 of the second electrode 12 and the second metal clip 341b, and is stored in the memory arranged in the substrate 34 or processed into data for output or communication by the signal processing unit.

According to another embodiment of the disclosure, instead of the first metal clip 341a and the second metal clip 341b, an electrically conductive metal terminal, for example, a metal brick or metal pogo pin, may be applied. Accordingly, the one end portion 111 of the first electrode 11 and the another end portion 122 of the second electrode 12 may be in contact with the metal brick or metal pogo pin electrically connected to the substrate 34.

The connection portion 13 is arranged between the first electrode 11 and the second electrode 12 and has a preset length.

The connection portion 13 insulates the first electrode 11 and the second electrode 12 from each other. The connection portion 13 is formed of a material having flexibility and elasticity, but does not include a metal powder having electrical conductivity. Accordingly, the connection portion 13 does not have electrical conductivity.

The first electrode 11 and the second electrode 12 are separated (spaced) by the connection portion 13, thereby obtaining clean (reliable) electric signals without interference.

The connection portion 13 is connected to the first electrode 11 and the second electrode 12.

The connection portion 13 includes one end portion 131 and another end portion 132 opposite to the one end portion 131. The connection portion 13 has a preset length extending from the one end portion 131 to the another end portion 132.

The one end portion 131 of the connection portion 13 is connected to the another end portion 112 of the first electrode 11. The another end portion 132 of the connection portion 13 is connected to the one end portion 121 of the second electrode 12.

According to an embodiment of the disclosure, the another end portion 112 of the first electrode 11 and the one end portion 131 of the connection portion 13 may be formed integrally with each other. Here, the term ‘integral’ means that the another end portion 112 of the first electrode 11 and the one end portion 131 of the connection portion 13 are connected as a single body without a physical disconnection, other than the another end portion 112 of the first electrode 11 and the one end portion 131 of the connection portion 13 being separately provided and connected in a welding or bonding manner. This may be achieved by double injection method. The another end portion 132 of the connection portion 13 and the one end portion 121 of the second electrode 12 may also be formed integrally with each other as described above.

According to another embodiment of the disclosure, the another end portion 112 of the first electrode 11 and the one end portion 131 of the connection portion 13 may be coupled (connected) by a method, such as heterogeneous silicon bonding. The another end portion 132 of the connection portion 13 and the one end portion 121 of the second electrode 12 may also be coupled (connected) by a method, such as heterogeneous silicon bonding.

The connection portion 13 is formed in a wavy shape 133. In other words, the connection portion 13 is formed in a winding (curvy) shape 133. Or, the connection portion 13 is formed in a plurally bent shape 133.

When the sleep care device 1 of the disclosure is worn on the ear, the connection portion 13 may be easily worn between the auricle and the head as the shape (wavy shape, winding shape, or plurally bent shape) 133 of the connection portion 13 is spread (unfolded). The shape 133 of the connection portion 13 returns to its original state after being worn, such that the ear hook 10 can be fully in contact (close contact) with the head and/or the auricle.

There is no need to replace the ear hook 10 depending on the user (specifically, the size of the auricle) by virtue of the shape 133 (wavy shape, winding shape, or plurally bent shape) of the connection portion 13 and the flexibility and elasticity of the shape 133, and thus one sleep care device 1 according to the disclosure can be used by several family members and other individuals.

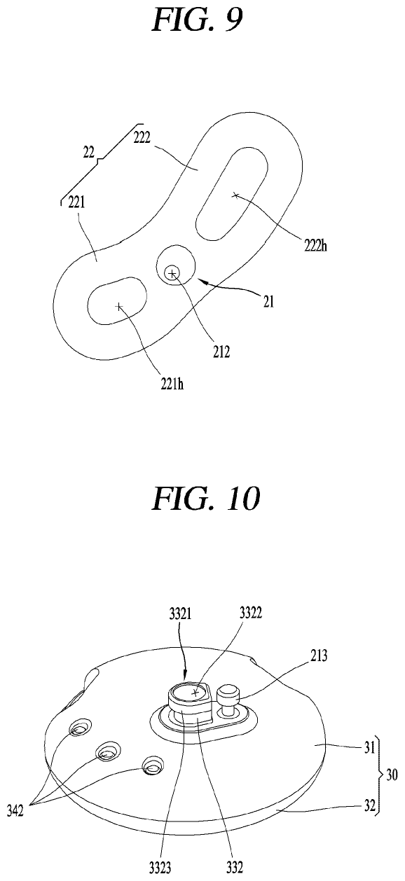

As illustrated in FIGS. 8 and 9, the eartip 20 comes into contact with the ear. Specifically, the eartip 20 is arranged to be in contact with the entrance of the external auditory canal (concha) of the ear.

The eartip 20 acts as an electrode itself and may detect and measure EEG signals. For example, the eartip 20 may be a measuring electrode that measures EEG signals.

The eartip 20 is connected to the housing 30.

The eartip 20 is arranged inside the auricle and comes into close contact with the skin.

The eartip 20 includes a tip body 21 and a tip ring 22.

The tip body 21 corresponds to a body of the eartip 20 and is connected to the housing 30.

The tip body 21 is arranged at the entrance to the external auditory canal of the ear.

The tip body 21 is not limited to a specific shape and may be formed in various shapes.

The tip body 21 includes a body groove 211 and a body hole 212.

The body groove 211 is a groove formed with a preset depth in one surface of the tip body 21. Specifically, the body groove 211 is formed concavely with a preset depth in one surface (e.g., a lower surface) of the tip body 21.

The body groove 211 may be at least one body groove. In FIG. 8, one body groove 211 is formed in the one surface of the tip body 21.

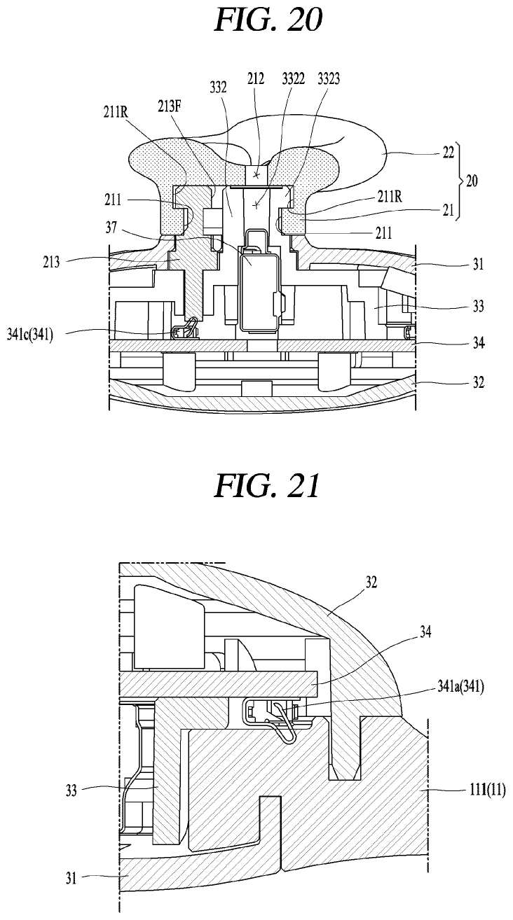

A stopping groove 211R (see FIG. 20) is dug in the body groove 211 in a direction orthogonal to a depth direction of the body groove 211.

The stopping groove 211R is formed in a side surface of the body groove 211 and is formed along a periphery of the side surface of the body groove 211.

One end portion of an electrical terminal (tip terminal) 213 to be described later is forcibly fitted into the stopping groove 211R. An upper end portion 3321 (see FIG. 17) of a speaker receiving part 332 formed in an inner housing 33 to be described later is forcibly fitted into the stopping groove 211R. The electrical terminal 213 and the speaker receiving part 332 are arranged adjacent to each other.

The upper end portion 3321 of the speaker receiving part 332 formed in the inner housing 33 to be described later is arranged in the body groove 211.

One end portion of the electrical terminal (tip terminal) 213 arranged in the housing 30 is located in the body groove 211.

As illustrated in FIG. 8, when one body groove 211 is formed in the tip body 21, the upper end portion 3321 of the speaker receiving part 332 and the one end portion of the electric terminal 213 are forcibly fitted into the one body groove 211. Accordingly, the eartip 20 is not easily separated from the housing 30 (specifically, the upper end portion 3321 of the speaker receiving part 332 and the one end portion of the electrical terminal 213). In the embodiment, the shape of the body groove 211 is not limited to a specific shape and may be formed in various shapes.

Although not shown, when two body grooves 211 are formed in the tip body 21, the upper end portion 3321 of the speaker receiving part 332 may be forcibly fitted into one body groove 211 and the one end portion of the electrical terminal 213 may be forcibly inserted into the other body groove 211. In the embodiment, the shape of the body groove 211 may be formed to correspond to the shape of the fitted upper end portion 3321 of the speaker receiving part 332 or the shape of the fitted one end portion of the electrical terminal 213.

The body hole 212 is formed in the body groove 211. Specifically, the body hole 212 is formed in a bottom surface of the body groove 211.

The body hole 212 is a hole that penetrates from the bottom surface of the body groove 211 to another surface (e.g., an upper surface) of the tip body 21.

The upper end portion 3321 of the speaker receiving part 332 formed in the inner housing 33 is arranged below the body hole 212 (see FIGS. 17 and 20).

Sound coming from a speaker 37 is transmitted to the outside of the sleep care device 1 of the disclosure sequentially through a sound pressure hole 3322, which is formed in the upper end portion 3321 of the speaker receiving part 332, and the body hole 212. When the body hole 212 is brought into close contact with the entrance of the external auditory canal of the ear, sound transmitted to the outside through the body hole 212 reaches the eardrum along the external auditory canal of the ear. Accordingly, the user who wears the sleep care device 1 of the disclosure on his or her ear can hear the sound coming from the sleep care device 1.

The tip ring 22 is connected to the tip body 21. Specifically, the tip ring 22 is connected to an upper side surface of the tip body 21.

The tip ring 22 is arranged inside the auricle and comes into close contact with the skin.

The tip ring 22 fixes the tip body 21. Accordingly, the tip body 21 placed at the entrance of the external auditory canal of the ear does not move or is not separated from the ear.

The tip ring 22 is formed in a ring shape with a central opening.

Two tip rings 22 (221 and 222) are arranged. The two tip rings 221 and 222 are respectively connected to one side and another side of the tip body 21. The two tip rings 221 and 222 each have a central opening 221h and 222h.

As illustrated in FIG. 8, the two tip rings 221 and 222 may be arranged asymmetrically and may also be formed in different shapes. This takes into account the fact that the inner shape of the ear is irregular. Accordingly, the two tip rings 221 and 222 can be easily placed inside the ear, and easily brought into contact with the skin.

Alternatively, although not shown, according to another embodiment of the disclosure, the two tip rings 221 and 222 may be arranged symmetrically and formed in the same shape. This may facilitate manufacturing of the two tip rings 221 and 222.

The eartip 20 may be formed of the same material as the ear hook 10 described above.

The eartip 20, i.e., the tip body 21 and the tip ring 22, are formed of a material having flexibility and elasticity. For example, the ear hook 10 may be formed of a silicone material having flexibility and elasticity.

The eartip 20 is formed of a material having flexibility and elasticity. Accordingly, the eartip 20 is arranged inside the auricle and maintains contact (close contact) with the skin.

The tip body 21 and tip ring 22 are deformed in shape by external force. The tip ring 22 may be deformed to fit the inner shape of the auricle, so that the tip body 21 can be placed at the entrance to the external auditory canal of the ear. This allows the user to clearly hear the sound transmitted through the body hole 212 while feeling comfortable wearing it, and maintains comfort even while wearing for a long time.

The two tip rings 22 may be easily deformed in shape by being formed of the material having the flexibility and elasticity and each being formed in a ring shape. Accordingly, even though the inner shape of the auricle including the concha is different depending on the user, the two tip rings 22 can be easily deformed to fit the inner shape of the auricle, allowing the eartip 20 to be easily placed and fixed in the user's ear. Therefore, the eartip 20 does not need to be replaced depending on the user, and one sleep care device 1 of the disclosure can be used by plural family members and other individuals.

As the two tip rings 22 are each formed in the ring shape, even if a part of the tip ring 22 does not come into contact with the skin of the ear, another part of the tip ring 22 comes into contact with the skin of the ear. Accordingly, when the sleep care device 1 of the disclosure is mounted on the ear, the eartip 20 is kept in contact with the skin of the ear.

The eartip 20 has the same electrical conductivity as the first electrode 11 and second electrode 12 of the ear hook 10 described above.

The eartip 20 itself acts as an electrode because the eartip 20 is electrically conductive. That is, in the disclosure, the eartip 20 is a type of electrode. Therefore, the eartip 20 may be named an ‘eartip electrode.’

The eartip 20, i.e., the tip body 21 and the tip ring 22, have electrical conductivity.

The tip body 21 and the tip ring 22 may be formed of a silicone material having flexibility and elasticity, and may be formed by including a metal powder having electrical conductivity in the silicone material.

Specifically, the tip body 21 and the tip ring 22 may be formed by mixing plate-shaped silver (Ag) powder, spherical silver (Ag) powder, and/or silver flakes into a silicone polymer. Accordingly, the first tip body 21 and the tip ring 22 have electrical conductivity while maintaining the characteristics of the silicon material. The eartip electrode has a resistance value of 10 Ω (ohm) of less.

The tip body 21 and the tip ring 22 act as an electrode and may detect and measure EEG signals. For example, the tip body 21 and the tip ring 22 may be a measuring electrode that measures EEG signals.

The eartip 20 (specifically, the tip body 21) is electrically connected to the electrical terminal 213 (which may be referred to as a ‘tip terminal 213’) arranged in the housing 30 (see FIG. 20). The eartip 20 (specifically, the tip body 21) is forcibly fitted into one end portion of the tip terminal 213.

The tip terminal 213 is formed in the shape of a metal rod having electrical conductivity.

The one end portion of the tip terminal 213 is connected to the eartip 20.

A terminal protrusion 213F (see FIG. 20) is formed on the one end portion of the tip terminal 213.

Specifically, the terminal protrusion 213F is formed on the one end portion of the tip terminal 213 and is formed along a periphery of the one end portion of the tip terminal 213.

The terminal protrusion 213F is formed to protrude in a direction orthogonal to a longitudinal direction of the tip terminal 213.

The terminal protrusion 213F is forcibly fitted into the stopping groove 211R formed in the tip body 21 (specifically, the body groove 211) of the eartip 20. Accordingly, the eartip 20 is not easily separated from the electrical terminal (tip terminal) 213.

Another end portion of the tip terminal 213 comes into contact with a metal clip 341 (this may be referred to as a ‘third metal clip 341c’), which is mounted on the substrate 34 (see FIG. 19).

The third metal clip 341c is mounted (fixed) to the electric circuit of the substrate 34 (see FIG. 20).

The third metal clip 341c is arranged between the another end portion 122 of the tip terminal 213 and the substrate 34, electrically connecting the another end portion 122 of the tip terminal 213 and the electric circuit of the substrate 34.

Specifically, one side (e.g., an upper portion) of the third metal clip 341c is in contact with the another end portion 122 of the tip terminal 213, and another side (e.g., a lower portion) of the third metal clip 341c is in contact with the electric circuit of the substrate 34.

Accordingly, EEG signals measured by the eartip 20 are transmitted to the substrate 34 via the tip terminal 213 and the third metal clip 341c, and stored in the memory arranged in the substrate 34 or processed into data for output or communication by the signal processing unit.

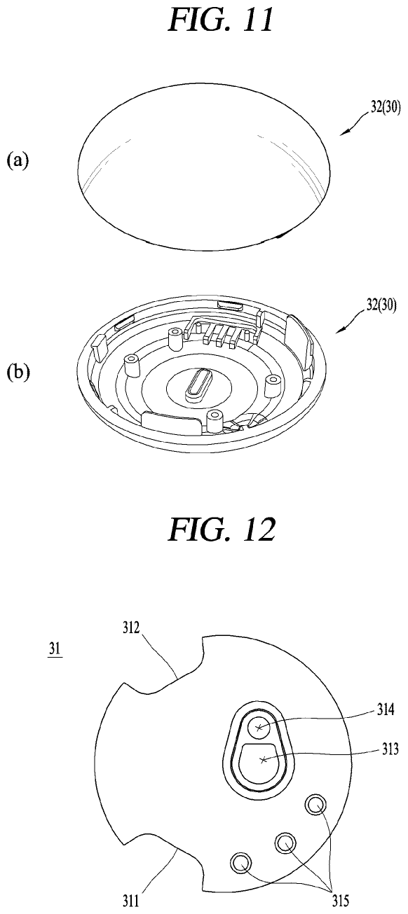

Referring to FIGS. 1 to 4 and FIGS. 10 to 15, the housing 30 forms the exterior of the sleep care device 1 of the disclosure.

The housing 30 is divided into an upper housing 32 and a lower housing 31.

As illustrated in FIG. 1, the upper housing 32 and the lower housing 31 may each be formed in a dome shape or a hemispherical shape. Here, a dome and hemisphere may be formed with a lower height (or depth) at the center.

The upper housing 32 and the lower housing 31 form a space inside. The upper housing 32 has a shape with a bottom open, and the lower housing 31 has a shape with a top open. For reference, FIG. 4 shows that the lower housing 31 to which the ear hook 10 and the eartip 20 are coupled is positioned at the top, and the upper housing 32 is positioned at the bottom.

The upper housing 32 is arranged to oppose the head with the lower housing 31 located therebetween. When the sleep care device 1 of the disclosure is worn on the user's ear, an outer surface of the upper housing 32 faces an opposite side of the head and is exposed to the outside.

A fastening member for coupling the upper housing 32 with the lower housing 31 may be formed on an inner surface of the upper housing 32, and fastening members for coupling with the substrate 34 or other members arranged inside the housing 30 and/or support members for supporting the substrate 34 or the other members may be formed on the inner surface of the upper housing 32.

The lower housing 31 is arranged to oppose the head with the eartip 20 located therebetween. When the sleep care device 1 of the disclosure is worn on the user's ear, an outer surface of the lower housing 31 opposes the ear.

One side 311 and another side 312 of the lower housing 31 are coupled to the one end portion 111 and the another end portion 122 of the ear hook 10.

The one side 311 and another side 312 of the lower housing 31 are spaced apart by a preset distance.

The one side 311 and another side 312 of the lower housing 31 are formed in a concave shape toward the center of the lower housing 31. The concave shape may correspond to the partial shapes of the one end portion 111 and another end portion 122 of the ear hook 10.

The lower housing 31 includes a speaker receiving part hole 313, through which the upper end portion 3321 of the speaker receiving part 332 formed in the inner housing 33 passes, and a tip terminal hole 314 through which the electric terminal (tip terminal 213) passes.

The speaker receiving part hole 313 may be formed to correspond to a cross-sectional shape of the speaker receiving part 332 arranged in the speaker receiving part hole 313.

A position of the speaker receiving part hole 313 in the lower housing 31 is not limited to a specific position and may correspond to a position where the speaker receiving part 332 of the inner housing 33 is arranged.

The tip terminal hole 314 may be formed to correspond to a cross-sectional shape of the tip terminal 213 arranged in the tip terminal hole 314.

A position of the tip terminal hole 314 in the lower housing 31 is not limited to a specific position and may correspond to a position where the tip terminal 213 is arranged.

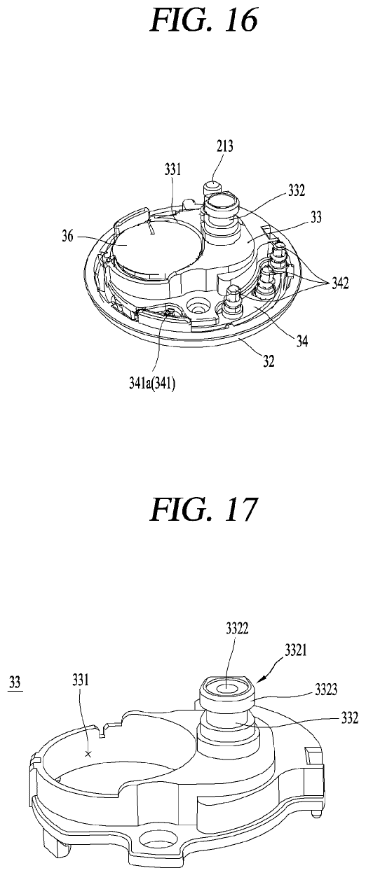

The sleep care device 1 of the disclosure includes at least one charging terminal 342 (see FIG. 16).

The charging terminal 342 is an electrical terminal for charging.

The charging terminal 342 is formed in the shape of a metal rod having electrical conductivity.

The lower housing 31 includes at least one charging terminal hole 315 through which the at least one charging terminal 342 passes.

The charging terminal 342 is arranged in the charging terminal hole 315 and exposed to the outside.

One end portion of the charging terminal 342 is exposed to the outside through the charging terminal hole 315, and another end portion of the charging terminal 342 is in contact with a metal clip 341 (this may be referred to as a ‘fourth metal clip 341d’), which is mounted on the substrate 34 and has electrical conductivity (see FIG. 19).

The fourth metal clip 341d is mounted (fixed) to the electric circuit of the substrate 34.

The fourth metal clip 341d is arranged between the another end portion 122 of the charging terminal 342 and the substrate 34, electrically connecting the another end portion 122 of the charging terminal 342 and the electric circuit of the substrate 34.

Specifically, one side (e.g., an upper portion) of the fourth metal clip 341d is in contact with the another end portion 122 of the charging terminal 342, and another side (e.g., a lower portion) of the fourth metal clip 341d is in contact with the electric circuit of the substrate 34.

The fourth metal clip 341d is arranged to match the number of the charging terminal 342.

The charging terminal hole 315 may be formed to correspond to a cross-sectional shape of the charging terminal 342 arranged in the charging terminal hole 315.

A position of the charging terminal hole 315 in the lower housing 31 is not limited to a specific position and may correspond to a position where the charging terminal 342 is arranged.

The lower housing 31 includes a fastening protrusion 3111 and a fastening screw groove 3112 formed in one side 311. The lower housing 31 includes a fastening protrusion 3121 and a fastening screw groove 3122 formed in another side 312.

The fastening protrusions 3111 and 3121 are formed to protrude from an inner surface of the one side 311 or another side 312 of the lower housing 31 toward the upper housing 32.

The fastening protrusion 3111 may be at least one fastening protrusion.

The fastening screw grooves 3112 and 3122 are formed in protrusion pillars 3113 and 3123 that protrude from the inner surface of the one side 311 or another side 312 of the lower housing 31 toward the upper housing 32. The fastening screw grooves 3112 and 3122 are formed concavely with a preset depth in surfaces (e.g., upper surfaces) of the protrusion pillars 3113 and 3123.

The housing 30 is formed by coupling the upper housing 32 and the lower housing 31.

The upper housing 32 and the lower housing 31 may be coupled in various ways, such as a bolt fastening manner, a screw fastening manner, a rotation coupling manner, and a hook coupling manner.

The bolt fastening manner is a manner of coupling the upper housing 32 and the lower housing 31 by, for example, forming a groove for bolt fastening in the upper housing 32, forming a through hole for bolt fastening through the lower housing 31, and fastening a bolt into the groove of the upper housing 32 through the through hole of the lower housing 31.

The screw coupling manner is a manner of coupling the upper housing 32 and the lower housing 31 by, for example, forming a screw thread along an edge of the upper housing 32, forming a screw groove along an edge of the lower housing 31, and rotating the screw thread of the upper housing 32 into the screw groove of the lower housing 31.

The rotation coupling manner is a manner of coupling the upper housing 32 and the lower housing 31 by, for example, forming at least one protrusion on an edge of the upper housing 32, forming at least one insertion groove in an edge of the lower housing 31, inserting the protrusion of the upper housing 32 into the insertion groove of the lower housing 31, and then rotating the protrusion of the upper housing 32.

The hook coupling manner is a manner of coupling the upper housing 32 and the lower housing 31 by, for example, forming at least one jaw on an edge of the upper housing 32, forming at least one groove in an edge of the lower housing 31, and inserting the jaw of the upper housing 32 into the groove of the lower housing 31.

In some embodiments, the sleep care device 1 according to an embodiment of the disclosure includes an inner housing 33 and a substrate 34.

The inner housing 33 and the substrate 34 are received inside the housing 30 (the upper housing 32 and the lower housing 31).

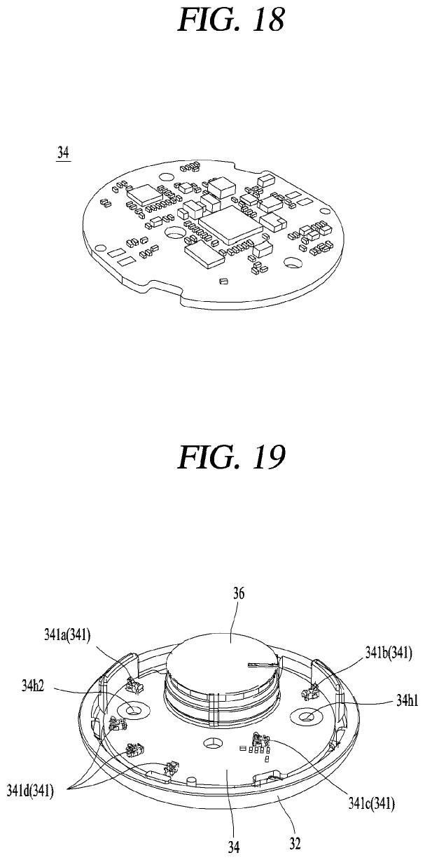

Referring to FIGS. 16 to 21, the inner housing 33 is arranged below the lower housing 31 in a state where the lower housing 31 of the sleep care device 1 of the disclosure opposes upward and the upper housing 32 opposes downward.

The inner housing 33 protects various electronic components mounted on the substrate 34.

The inner housing 33 includes a battery receiving part 331 and a speaker receiving part 332.

The battery receiving part 331 receives a battery 36 to be described later.

The battery receiving part 331 may be formed as a through hole for receiving the battery 36 as illustrated in the drawing. However, the battery receiving part 331 is not limited to the through hole and may be formed in various shapes.

The speaker receiving part 332 receives a speaker 37 to be described later.

The speaker receiving part 332 is formed in an upper surface of the inner housing 33.

The speaker receiving part 332 may be formed to correspond to the shape of the speaker 37.

The upper end portion 3321 of the speaker receiving part 332 penetrates the speaker receiving part hole 313 of the lower housing 31 and is arranged outside the lower housing 31.

The upper end portion 3321 of the speaker receiving part 332 is forcibly fitted into the tip body 21 (specifically, the body groove 211) of the eartip 20.

The upper end portion 3321 of the speaker receiving part 332 includes a side protrusion 3323 that protrudes in a direction orthogonal to a direction toward the lower housing 31.

The side protrusion 3323 may be formed continuously along a side surface of the upper end portion 3321 of the speaker receiving part 332, or may be formed as a plurality of side protrusions 3323 on the side surface of the upper end portion 3321 of the speaker receiving part 332.

The side protrusion 3323 is forcibly fitted into the tip body 21 of the eartip 20 (specifically, the body groove 211 formed in the tip body 21). Specifically, the side protrusion 3323 formed on the upper end portion 3321 of the speaker receiving part 332 is forcibly fitted into the stopping groove 211R formed in the body groove 211 of the tip body 21.

A sound pressure hole 3322 is formed in the upper end portion 3321 of the speaker receiving part 332. Specifically, the sound pressure hole 3322 is formed in an upper surface of the upper end portion 3321 of the speaker receiving part 332.

The sound pressure hole 3322 is a through hole that penetrates the upper surface of the upper end portion 3321 of the speaker receiving part 332.

The sound pressure hole 3322 communicates with the body hole 212 formed in the tip body 21 of the eartip 20 when the upper end portion 3321 of the speaker receiving part 332 is forcibly fitted into the tip body 21 of the eartip 20.

When sound is output from the speaker 37 received in the speaker receiving part 332, the sound passes through the sound pressure hole 3322 formed in the upper end portion 3321 of the speaker receiving part 332 and the body hole 212 formed in the tip body 21 of the eartip 20, and is then transmitted to the outside of the sleep care device 1 of the disclosure. The sound reaches the eardrum through the external auditory canal.

The speaker receiving part 332 is formed in an upper surface of the inner housing 33.

A through hole through which the electrical terminal (tip terminal) 213 passes is formed through the upper surface of the inner housing 33. The through hole is arranged adjacent to the speaker receiving part 332. The tip terminal 213 is arranged in the through hole. One end portion of the tip terminal 213 is forcibly fitted into the tip body 21 of the eartip 20 and another end portion of the tip terminal 213 is electrically connected to the substrate 34 via the metal clip 341c.

The shape of the inner housing 33 is not limited to a specific shape, and may be formed in various ways considering the shapes or functions of the battery 36 received inside the inner housing 33, the speaker 37, and various electronic components mounted on the substrate 34.

The inner housing 33 may be fastened to the lower housing 31 or the upper housing 32 by fastening means, such as a rivet, screw, or clip.

The substrate 34 refers to a printed circuit board.

Various electronic components are mounted on the substrate 34. Here, the various electronic components include electronic components related to storing EEG signals measured from the eartip 20 (a memory), converting analog signals into digital signals (a signal processing unit), transmitting data to or receiving data from a mobile terminal (a communication unit), transmitting sound through the speaker 37 (a speaker), outputting various information to a display unit when the sleep care device 1 of the disclosure includes a display unit that displays various information, outputting sound to be transmitted through the speaker (an output unit), and controlling signals and data so that the above-described functions are organically performed (a controller).

The substrate 34 may be fastened to the upper housing 32 by fastening means, such as a screw or rivet.

A plurality of metal clips 341 are arranged on the substrate 34, and a plurality of fastening screw holes 34h1 and 34h2 are formed in the substrate 34.

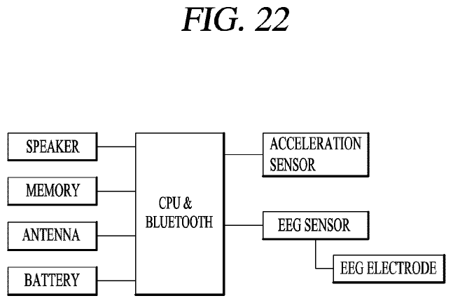

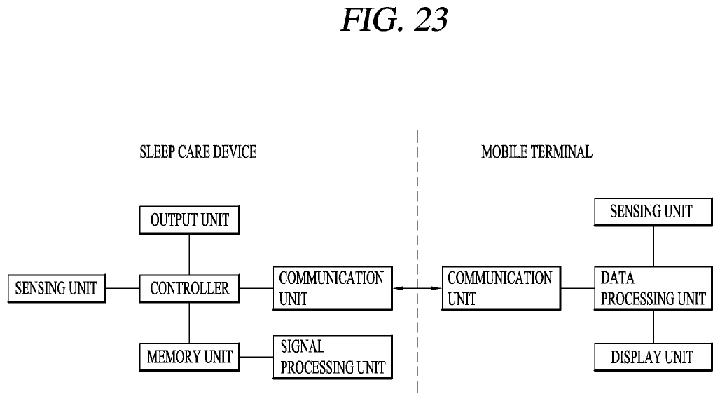

Referring to FIGS. 4, 16, 19, 20, 22, and 23, the sleep care device 1 of the disclosure includes a speaker 37, a memory, an antenna 35, a battery 36, an acceleration sensor, an EEG sensor, an EEG electrode, and a central processing unit (CPU & Bluetooth).

The speaker 37 is a device that converts an electric acoustic signal into a sound wave.

The speaker 37 is built into the housing 30. Specifically, the speaker 37 is received in the speaker receiving part 332 of the inner housing 33 and is electrically connected to the substrate 34.

The speaker 37 includes a series of parts and accessories necessary to output sound.

Sound coming from the speaker 37 passes through the sound pressure hole 3322 formed in the speaker receiving part 332 and the body hole 212 formed in the tip body 21 of the eartip 20, and is then transmitted to the outside of the sleep care device 1 of the disclosure.

The sound from the speaker 37 may be output by the output unit.

The memory is arranged on the substrate 34 and corresponds to a type of semiconductor memory chip that stores various types of data.

The memory stores EEG signals (e.g., analog signals) measured by the eartip 20, digital signals processed by the signal processing unit, or various types of information for display.

The antenna 35 is a communication device for transmitting and receiving various types of data.

The antenna 35 may be a Bluetooth antenna.

The antenna 35 is electrically connected to the substrate 34. Electronic components for communication are mounted on the substrate 34.

The sleep care device 1 of the disclosure may be linked to a mobile terminal.

Data processed in the communication unit of the sleep care device 1 may be transmitted to the mobile terminal via the antenna 35. Data transmitted from a communication unit of the mobile terminal may be received by the sleep care device 1 of the disclosure through the antenna 35.

Data (e.g., sound) transmitted through the communication unit of the mobile terminal is received through the communication unit of the sleep care device 1 of the disclosure, and the data may be output through the output unit under the control of the controller. The output data (sound) is transmitted to the outside of the sleep care device 1 through the speaker.

The battery 36 stores electricity and supplies driving electricity necessary to drive the sleep care device 1 of the disclosure.

The battery 36 is received in the battery receiving part 331 of the inner housing 33.

The battery 36 is electrically connected to the charging terminal 342 and the substrate 34.

Specifically, the charging terminal 342 is electrically connected to the substrate 34 via the metal clip 341 (the fourth metal clip 341d), and the battery 36 is electrically connected to the substrate 34. The charging terminal 342 is electrically connected to the battery 36 through the metal clip 341 and an electric circuit printed on the substrate 34.

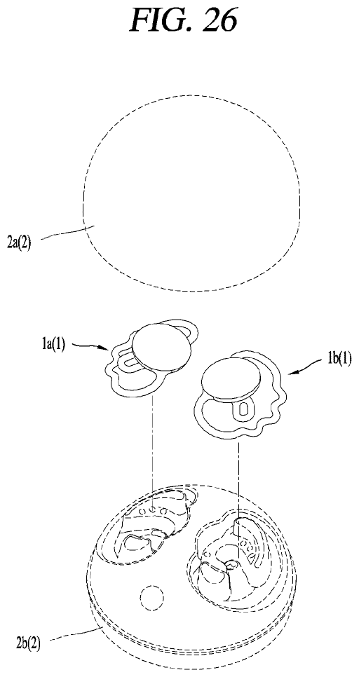

The sleep care device 1 of the disclosure may be charged by being placed on a charging case. FIG. 26 is a view of an embodiment in which the sleep care device 1 of FIG. 1 is placed in the charging case 2. The charging case 2 illustrated in FIG. 26 has an arbitrary shape and may be formed into a different shape.

The charging case 2 may include an upper case 2a and a lower case 2b.

The lower case 2b includes a pair of mounting parts. A pair of sleep care devices 1a and 1b to be worn on the right and left ears, respectively, are placed in the pair of mounting parts.

When the sleep care device 1 of the disclosure is placed in the charging case 2, the charging terminal arranged in the charging case 2 and the charging terminal 342 of the sleep care device 1 come into contact with each other, and electricity is stored in the battery 36 embedded in the sleep care device 1 through the charging terminal of the charging case 2 and the charging terminal 342 of the sleep care device 1.

The electricity stored in the battery 36 becomes driving electricity for driving the sleep care device 1 of the disclosure when the sleep care device 1 is operated by a user (or by an operating signal from the controller).

The charging case 2 is also a storage case in which the sleep care device 1 of the disclosure is stored.

The acceleration sensor is a device that processes output signals to measure dynamic forces, such as acceleration, vibration, and impact of an object.

The acceleration sensor may be a commonly used 3-axis gyro sensor and/or 3-axis acceleration sensor.

The acceleration sensor may detect an object's motion state in detail, leading to a wide range of applications for various purposes. A detailed description of the acceleration sensor itself will be omitted.

The acceleration sensor may be mounted on the substrate 34 or arranged inside the housing 30 and electrically connected to the substrate 34.

The sleep care device 1 of the disclosure may determine the user's activity by detecting the direction and speed that the user moves using the acceleration sensor.

For example, in the state where the sleep care device 1 of the disclosure with the embedded acceleration sensor is worn on the user's ear, when the user is standing, lying down, or moves his or her head while lying down, the sleep care device 1 of the disclosure may determine the user's movement state from a signal output from the acceleration sensor.

Accordingly, the sleep care device 1 of the disclosure can determine whether the user is in an active state (Wake), a sleeping state, a REM sleep state, a light sleep state, or a deep sleep state by using signals output from the acceleration sensor and EEG signals output from the eartip 20.

The EEG sensor is mounted on the substrate 34 and corresponds to a type of semiconductor chip.

The EEG sensor is used to receive, amplify, and filter EEG signals output from the ear hook 10 and the eartip 20.

For example, the EEG sensor obtains a difference between an EEG signal measured by the reference electrode (the first electrode 11 or the second electrode 12) of the ear hook 10 and an EEG signal measured by the eartip (the eartip electrode) 20, and amplifies the difference.

The amplified analog signal is filtered and then transmitted to the signal processing unit. The signal processing unit converts the filtered analog signal into a digital signal. The digital signal includes EEG signals for each frequency band.

The EEG electrodes correspond to the first electrode 11 and the second electrode 12 formed on the ear hook 10, and the eartip electrode.

In the disclosure, the first electrode 11 may be a reference electrode, the second electrode 12 may be a ground electrode (bias), and the eartip electrode may be a measuring electrode. However, according to another embodiment of the disclosure, the second electrode 12 or the eartip electrode may be the reference electrode, the first electrode 11 or the eartip electrode may be the ground electrode (bias), or the first electrode 11 or the second electrode 12 may be the measuring electrode.

The specific descriptions of the first electrode 11, the second electrode 12 and the eartip electrode have been given, specific descriptions of the electrodes are omitted.

The central processing unit (CPU & Bluetooth) is mounted on the substrate 34 and corresponds to a type of semiconductor chip.

The central processing unit interprets program commands, calculates and processes data, outputs the results, and controls the output results to be executed. The central processing unit includes a Bluetooth function.

The Bluetooth refers to a short-range wireless technology standard that connects portable devices, such as a mobile phone, a laptop computer, an earphone, and a headphone, to enable information exchange. The Bluetooth is mainly used when a low-power wireless connection is required at an ultra-short distance of about 10 meters.

The sleep care device 1 of the disclosure includes the Bluetooth function and the speaker 37, so that sound from a mobile phone is transmitted to the user without a cable.

The sleep care device 1 according to an embodiment of the disclosure includes a sensing unit, a memory unit, a signal processing unit, a communication unit, an output unit, and a controller.

The sensing unit detects and measures the user's EEG signals.

The sensing unit includes the ear hook 10 and the eartip 20 described above. Specifically, the sensing unit includes the first electrode 11, the second electrode 12 formed in the ear hook 10, and the eartip 20 (the eartip electrode).

The first electrode 11, the second electrode 12, and the eartip electrode serve as sensors that detect and measure EEG signals.

Accordingly, the sensing unit can detect and measure the user's EEG signals using the first electrode 11, the second electrode 12, and the eartip electrode.

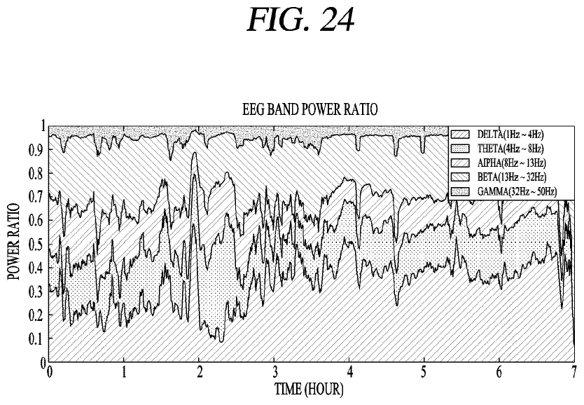

FIG. 24 is a view of EEG signals measured by the sleep care device 1 according to the disclosure. It was experimentally verified whether the sleep care device 1 of the disclosure measures EEG signals.

The sleep care device 1 of the disclosure was used to measure bio-signals from the user's ear. Here, the user's ear indicates the user's ear in contact with the sleep care device 1 of the disclosure and surroundings of the ear.

By analyzing the bio-signals measured by the sleep care device 1 of the disclosure for each frequency band, it was confirmed that the bio-signals measured by the sleep care device 1 of the disclosure are observed within a characteristic frequency band of EEG, namely, 1 Hz to 50 Hz, for each frequency band.

Accordingly, it was confirmed that the bio-signals measured by the sleep care device 1 of the disclosure include the EEG signals.

Therefore, the user's sleep stage can be determined using the EEG signals (specifically, signals for each frequency band over time) measured by the sleep care device 1 of the disclosure.

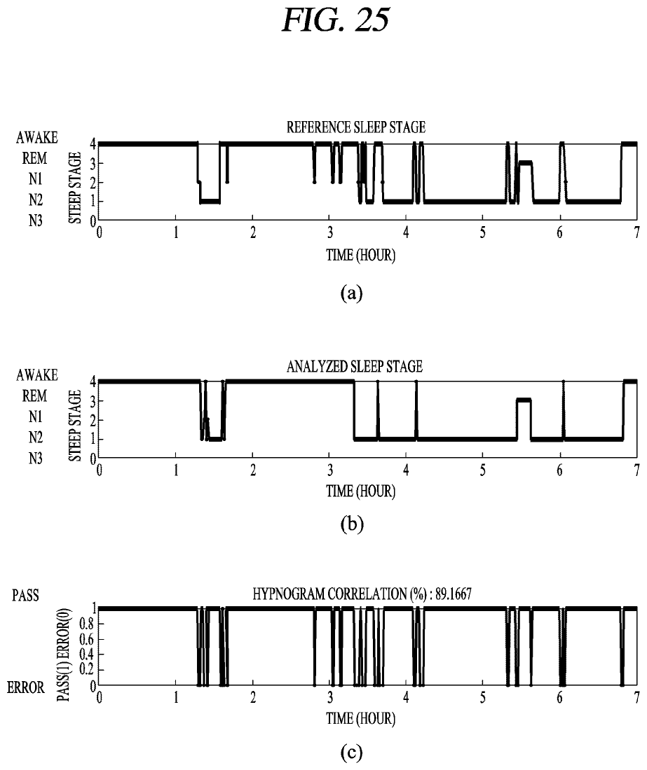

FIG. 25 compares EEG measured from the ear and EEG measured from the scalp using the sleep care device 1 according to the disclosure. (a) of FIG. 25 shows EEG measured from the scalp, (b) of FIG. 25 shows EEG measured from the ear, and (c) of FIG. 25 shows similarity between the two measurements. Here, the ear indicates the user's ear in contact with the sleep care device 1 of the disclosure and surroundings of the ear.

To verify the accuracy of the EEG measured by the sleep care device 1 of the disclosure, the EEG measured by the sleep care device 1 was compared with the EEG measured from the scalp.

During a polysomnographic sleep study, EEG signals were measured simultaneously from the scalp and ear, and compared with previously confirmed sleep stages.

Similarity of 89.1% was observed as a result of comparing the EEG signals measured by the sleep care device 1 of the disclosure with the confirmed sleep stages.

Accordingly, the sleep care device 1 of the disclosure can measure EEG signals from the user's ear and determine the user's sleep stage from the measured EEG signals.

The sensing unit may obtain bio-signals, such as body temperature, heart rate, and oxygen saturation in addition to the EEG signals, and transmit the obtained results to the signal processing unit by the controller.

The sensing unit is electrically connected to the controller, and detects and measures the user's EEG signals by a control signal of the controller.

The EEG signals measured in the sensing unit are transmitted to the memory unit or signal processing unit by the controller.

The memory unit includes the above-described memory.

The memory unit is electrically connected to the controller. The memory unit may store EEG signals (e.g., analog signals) measured by the eartip 20 in the memory by a control signal from the controller, store digital signals processed by the signal processing unit in the memory, or store various types of information for display and sound signals output through the speaker in the memory.

The signal processing unit converts the EEG signals (the analog signals) input from the sensing unit into digital signals.

The signal processing unit may process signals to be transmitted to a mobile terminal via the communication unit, or may process various signals, such as sounds and the like, to be output to the outside via the output unit.

The communication unit may include at least one module that enables wireless communication between the sleep care device 1 of the disclosure and the mobile terminal, and between the sleep care device 1 and the charging case.

The communication unit may include at least one of a broadcast receiving module, a mobile communication module, a wireless Internet module, a short-range communication module, and a location information module.

The communication unit is controlled by the controller and includes an antenna 35.

The communication unit may transmit various data to the mobile terminal or receive various data from the mobile terminal.

The output unit may include a display unit that displays various types of information, and output various types of information to the display unit. The display unit includes various electronic components for displaying information.

The output unit includes a speaker and may output sound through the speaker. The speaker includes various electronic components for outputting sound.

The output unit is controlled by the controller.

The output unit may output sound received from the mobile terminal or sound stored in the memory unit through the speaker by a control signal of the controller.

The controller includes a central processing unit (CPU & Bluetooth).

The controller controls the sensing unit, the memory unit, the signal processing unit, the output unit, and the communication unit described above.

The controller may provide or process information or sound appropriate for the user by processing signals, data, and information that are input or output through the sensing unit, the memory unit, the signal processing unit, the output unit, and the communication unit, or by operating application programs stored in the controller or the memory unit.

The controller may control the battery 36 to turn on/off the sleep care device 1 of the disclosure when the user's input signal is received. Here, the user's input signal may indicate, for example, an action that the user presses a button located on the sleep care device 1 or the mobile terminal or an action of coupling or separating the sleep care device to and from the charging case.

The sleep care device 1 according to an embodiment of the disclosure may be operated in conjunction with a mobile terminal.

The mobile terminal includes devices, such as mobile phones, smart phones, laptop computers, digital broadcasting terminals, personal digital assistants (PDAs), portable multimedia players (PMPs), navigation devices, slate PCs, tablet PCs, or ultrabooks, smartwatches, and the like.

The mobile terminal may include a communication unit, a sensing unit, a data processing unit, and a display unit.

The communication unit of the mobile terminal may include at least one module that enables wireless communication between the sleep care device 1 of the disclosure and the mobile terminal, and between the mobile terminal and the charging case.

The communication unit of the mobile terminal may include at least one of a broadcast receiving module, a mobile communication module, a wireless Internet module, a short-range communication module, and a location information module.

The communication unit of the mobile terminal is controlled by a controller and includes an antenna.

The communication unit of the mobile terminal may receive data transmitted from the communication unit of the sleep care device 1, and may also transmit data processed by the data processing unit of the mobile terminal to the communication unit of the sleep care device 1.

The sensing unit of the mobile terminal may detect the user's snoring sound, a breathing pattern using a radio frequency (RF), illuminance, and the like.

The sensing unit of the mobile terminal may not come into contact with the user's body.

The data processing unit of the mobile terminal may generate parameters for controlling the output unit using bio-signals (e.g., EEG signals) transmitted from the sleep care device 1.

The data processing unit of the mobile terminal may use signals received through the communication unit to generate parameters for controlling the output unit of the sleep care device 1, and some of the parameters may be displayed on the display unit. The parameters may be transmitted in real time to the communication unit of the sleep care device 1 through the communication unit of the mobile terminal, and may be utilized by the controller of the sleep control device 1 to control the output unit.

The display unit of the mobile terminal may display results processed by the data processing unit. The display unit includes various electronic components for displaying information.

The mobile terminal may further include a controller.

The controller of the mobile terminal may control the communication unit, the sensing unit, the data processing unit, and the display unit of the mobile terminal.

The controller of the mobile terminal may provide the user with or process appropriate information or sounds by processing signals, data, and information input or output through the communication unit, the sensing unit, the data processing unit, and the display unit of the mobile terminal, or by operating application programs stored in the controller or memory.

In this way, the sleep care device according to an embodiment of the disclosure can be suppressed from being easily separated from the user's body during the user's sleep state and can measure reliable EEG signals.

The sleep care device according to an embodiment of the disclosure can enable the user to feel comfortable wearing the sleep care device while sleeping.

The sleep care device according to an embodiment of the disclosure can be used by plural family members and other individuals without needing to replace the ear hook and the eartip depending on users.

Features, structures, effects, and the like described in those embodiments are included in at least one embodiment of the disclosure, and are not necessarily limited to only one embodiment. Furthermore, features, structures, effects, and the like illustrated in each embodiment may be combined or modified with respect to other embodiments by those skilled in the art to which the embodiments belong. Therefore, contents related to such combinations and modifications should be construed as being included in the scope of the disclosure.

In addition, the foregoing description has been made with reference to the embodiments, but it is merely illustrative and is not intended to limit the disclosure. It will be apparent that other changes and applications can be made by those skilled in the art to which the disclosure belong without departing from substantial features of the embodiments of the disclosure. For example, each component specifically shown in the embodiments may be modified and practiced. And it should be construed that differences relating to such changes and applications are included in the scope of the disclosure defined in the appended claims.

### Description of Reference Numerals

1: Sleep care device 2: Charging Case
10: Ear hook 11: First electrode
12: Second electrode 13: Connection portion
20: Eartip 21: Tip body
22: Tip Ring 30: Housing
31: Lower Housing 32: Upper housing
33: Inner Housing 34: Substrate
35: Antenna 36: Battery
37: Speaker

What is claimed is:

1. A sleep care device mounted on a user's ear, the sleep care device comprising:
a housing forming an exterior of the sleep care device;
an ear hook connected to the housing to form a closed loop, arranged between the user's head and an auricle, and comprising an electrode having electrical conductivity; and
an eartip connected to the housing, arranged at an entrance of an external auditory canal, and having electrical conductivity,
wherein the ear hook and the eartip are made of a material having flexibility and elasticity and measure the user's electroencephalogram (EEG).

2. The sleep care device of claim 1, wherein the ear hook comprises a first electrode and a second electrode having electrical conductivity, and a connection portion arranged between the first electrode and the second electrode.

3. The sleep care device of claim 2, wherein the first electrode or the second electrode is formed in an arcuate shape.

4. The sleep care device of claim 2, wherein the connection portion is formed in a wavy shape.

5. The sleep care device of claim 2, wherein a plurality of fastening holes are formed in each of one end portion of the first electrode and another end portion of the second electrode.

6. The sleep care device of claim 5, wherein the plurality of fastening holes comprise at least one fastening protrusion hole and a fastening screw hole.

7. The sleep care device of claim 6, wherein the housing comprises an upper housing and a lower housing,
a fastening protrusion formed on one side of the lower housing is arranged by being inserted into the fastening protrusion hole formed in the one end portion of the first electrode, and
a fastening protrusion formed on another side of the lower housing is arranged by being inserted into the fastening protrusion hole formed in the another end portion of the second electrode.

8. The sleep care device of claim 6, wherein the housing comprises an upper housing and a lower housing,
the fastening screw hole formed in the one end portion of the first electrode is placed in a fastening screw groove formed in one side of the lower housing, and
the fastening screw hole formed in the another end portion of the second electrode is placed in a fastening screw groove formed in another side of the lower housing.

9. The sleep care device of claim 2, wherein a substrate is arranged inside the housing, and
one end portion of the first electrode and another end portion of the second electrode are electrically connected to the substrate by a metal clip mounted on the substrate and having electrical conductivity.

10. The sleep care device of claim 1, wherein the eartip comprises a tip body connected to the housing and a tip ring connected to the tip body.

11. The sleep care device of claim 10, wherein the tip body comprises a body groove and a body hole,
the body groove is a groove formed with a preset depth in one surface of the tip body, and
the body hole is a hole penetrating from a bottom surface of the body groove to another surface of the tip body.

12. The scroll compressor of claim 11, wherein a substrate is arranged inside the housing, and
one end portion of an electrical terminal electrically connected to the substrate is forcibly fitted into the body groove.

13. The sleep care device of claim 11, wherein the housing comprises an upper housing and a lower housing,
a substrate is arranged between the upper housing and the lower housing,
an inner housing is arranged between the lower housing and the substrate,
an upper end portion of a speaker receiving part formed in the inner housing is forcibly fitted into the body groove, and
the upper end portion of the speaker receiving part is arranged below the body hole.

14. The sleep care device of claim 13, wherein a sound pressure hole communicating with the body hole is formed in the upper end portion of the speaker receiving part.

15. The sleep care device of claim 10, wherein the tip ring has a ring shape and is arranged as two tip rings, and
the two tip rings are respectively connected to one side and another side of the tip body.

16. The sleep care device of claim 1, wherein an electrode of the ear hook and the eartip are formed by mixing plate-shaped silver (Ag) powder, spherical silver (Ag) powder, or silver flakes with a silicone polymer.

17. The sleep care device of claim 1, wherein the housing comprises an upper housing arranged toward an opposite side of the user's head, and a lower housing arranged to oppose the head with the eartip located therebetween,
a substrate is arranged between the upper housing and the lower housing,
an inner housing is arranged between the lower housing and the substrate, and
the lower housing comprises a speaker receiving part hole through which an upper end portion of a speaker receiving part formed in the inner housing passes, and a tip terminal hole through which an electrical terminal electrically connected to the substrate passes.
